(19) United States
(12) Patent Application Publication
(10) Pub. No.: US 2025/0057298 A1
(43) Pub. Date: Feb. 20, 2025

(54) FALSE NAIL REMOVAL DEVICE

(71) Applicant: ENNIO PRODUCTS INC., Foshan (CN)

(72) Inventors: Shouji ZHAO, Foshan (CN); Ying SUN, Foshan (CN)

(73) Assignee: ENNIO PRODUCTS INC., Foshan, GD (CN)

(21) Appl. No.: 18/628,830

(22) Filed: Apr. 8, 2024

(30) Foreign Application Priority Data
Aug. 18, 2023 (CN) 202322230966.1

(51) Int. Cl. A45D 29/17; A45D 34/04

(52) U.S. Cl. CPC A45D 29/17; A45D 34/04

(57) ABSTRACT

A false nail removal device includes a holding part and a nail removing part arranged at one end of the holding part. The nail removing part is made of a hard material which is convenient for inserting nails and artificial nails. Compared with the prior art, the technical solution of the present application has the beneficial effects that: compared with the brushes and soft materials in the prior art, the nail removing part of the present application is made of a hard material, which can be inserted into the gap between the nail and the artificial nail. The nail removing part of the present application is designed with a slope which is easily to be inserted into a gap between a person nail and an artificial nail, whereby user comfortability is improved during the process of removing the artificial nails/nail gels from person's nails.

### TECHNICAL FIELD

The present application specifically relates to the technical field of nail beauty products, and in particular to a false nail removal device.

### BACKGROUND

With the development of society, people, especially women, pay more attention to their own decoration and dressing. Nowadays, many fashionable ladies like to beautify their nails, and even add patterns or ornaments to their nails by means of painting to enhance their image. Therefore, nail art has become more and more popular among ladies.

At present, fashionable ladies generally use the method of soaking false nail removing liquid to remove false nails, but because false nail removing liquid is made of chemical materials, it has a certain stimulating and damaging effect on people's fingernails, and when the soaking time is too long, the damage to people's fingernails will be greater. Then, some consumers often use some auxiliary tools to speed up the removal of artificial nails or nail polish, and often remove nail gel on nails in the following two ways: the first method is to push the softened nail gel off the nails with a metal push rod; the second method is to polish the nail gel off the nail with a special nail file. However, in the actual operation, it is found that both methods have some defects: 1, in the process of removing the nail gel, the metal push rod will exert a certain force on the oblique angle of the nail surface, and the thin and soft nail is easy to feel uncomfortable or even painful; at the same time, when removing the nail gel, the shovel head of the push rod is easy to push down the cuticle of the nail together with the nail gel, resulting in the thinning and damage of the nail surface, and the whole nail removal operation will take a certain time; 2, professional nail file and grinding will have certain requirements for the proficiency of operation techniques, especially for novice operators, and it is easy to grind nails if the method is wrong, which embodies the shortcomings of cumbersome operation and slow nail gel removal.

Thus, there is also a nail removing bottle with a brush in the market, which is used for manual nail removal. The nail removing bottle requires the hand to be put in the bottle body and soaked in nail removing liquid, and the fingers should be rotated, so that the brush can rub the nail surface to achieve the nail removal effect. Although the nail removing bottle has a better nail removal effect than the above-mentioned method, the nail removing bottle with this structure also has disadvantages such as complicated operation, time-consuming and laborious work, low nail removal speed, not good nail removal effect, and even damage to the hands.

### SUMMARY

In order to achieve the above purpose, the present application provides a false nail removal device.

A false nail removal device comprises a holding part and a nail removing part, wherein the nail removing part is arranged at an end of the holding part, and the nail removing part is made of a hard material so that the nail removing part can be inserted between a person's nail and a false nail mounted on the person's nail.

Preferably, the nail removing part is provided with a channel.

Preferably, an end of the nail removing part opposite to the holding part is provided with a slope.

Preferably, the holding part is provided with a cavity for storing a nail removing liquid.

Preferably, the holding part is provided with a pressing device.

Preferably, a linkage device is arranged between the cavity and the nail removing part.

Preferably, the false nail removal device comprises a cap covered on the nail removing part.

Compared with the prior art, the technical solution of the present application has the beneficial effects that:
1. Compared with the brush and soft material in the prior art, the nail removing part of the present application is of a hard material, which can be inserted into the gap between the nail and the artificial nail.
2. The nail removing part of the present application is designed with an inclined plane which is convenient to insert into the gap between a person's nail and the artificial nail/nail gel.
3. The slope with an inclined plane facilitates the tip of the nail removing part to be inserted into gaps between person's nails and artificial nails/nail gels, whereby user comfortability is improved during the process of removing the artificial nails/nail gels from person's nails.

### BRIEF DESCRIPTION OF DRAWINGS

FIG. 1 is a side perspective view of the false nail removal device of the present application.

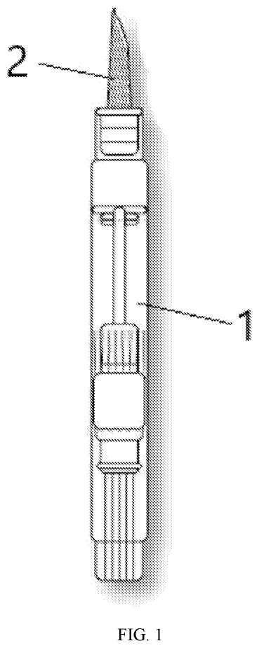

FIG. 2 is the structural schematic diagram of the false nail removal device of the present application.

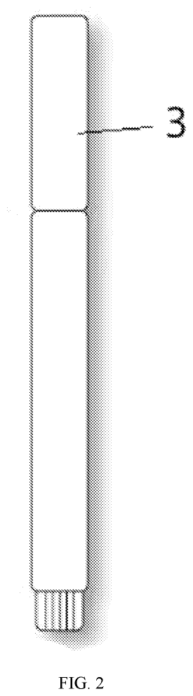

FIG. 3 is a front perspective view of the false nail removal device of the present application.

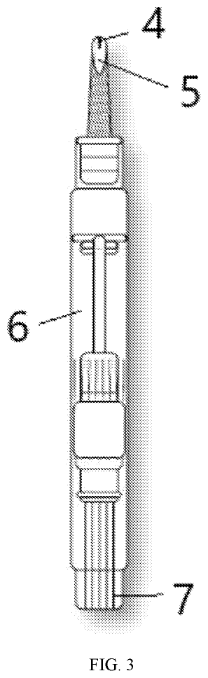

FIG. 4 is the second structural schematic diagram of the false nail removal device of the present application.

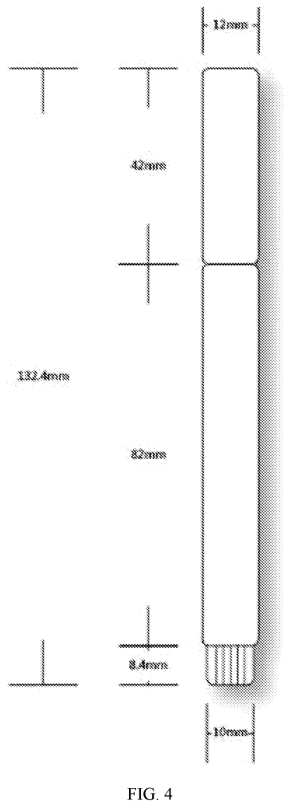

FIG. 5 is the second side perspective view of the false nail removal device of the present application.

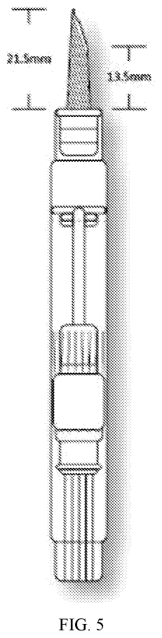

FIG. 6 is a left side view of the false nail removal device of the present application.

FIG. 7 is a right side view of the false nail removal device of the present application.

FIG. 8 is a front view of the false nail removal device of the present application.

FIG. 9 illustrates the holding part of the false nail removal device with a pressing device.

FIG. 10 illustrates a linkage device connected between the cavity and the removing part.

In the figures: 1, Holding part; 2, Nail removing part; 3, Cap; 4, Channel; 5, Slope; 6, Cavity; 7, Pressing device; 8, linkage device

### DETAILED DESCRIPTION

In order to clearly explain the technical characteristics of this solution, the present application will be described in detail through specific embodiments with reference to the drawings. The following disclosure provides multiple different embodiments or examples to realize different structures of the present application. In order to simplify the disclosure of the present application, the components and arrangements of specific examples are described below. In addition, the present application can repeatedly refer to numbers and/or letters in different examples. This repetition is for simplicity and clarity, and does not in itself indicate the relationship between the various embodiments and/or arrangements discussed. It should be noted that the components illustrated in the drawings are not necessarily drawn to scale. The present application omits the description of well-known components and processing technologies and processes to avoid unnecessarily limiting the present application.

A false nail removal device includes a holding part 1 and a nail removing part 2. The nail removing part 2 is arranged at one end of the holding part 1, and the nail removing part 2 is made of a hard material for example PP (Polypropylene) material so that it can be easily inserted into a gap between a person's nail and a painted/artificial nail attached on the person's nail.

In a preferred embodiment, the nail removing part 2 is provided with a channel 4. The channel 4 is used for nail removing liquid to pass through.

In a preferred embodiment, one end of the nail removing part 2 far away from the holding part 1 is provided with a slope 5 which facilitates the nail removing part 2 to be inserted into gaps between nails and artificial nails. The slope 5 with an inclined plane facilitates the nail removing part 2 to be inserted into gaps between person's nails and artificial nails, whereby user comfortability is improved during the process of removing the artificial nails from person's nails.

In a preferred embodiment, the holding part 1 is provided with a cavity 6 for storing the nail removing liquid.

In a preferred embodiment, the holding part 1 is provided with a pressing device 7.

In a preferred embodiment, a linkage device is arranged between the cavity 6 and the nail removing part 2. The amount of the nail removing liquid reaching nails is small and scattered, which is beneficial to nail removal and hand cleaning.

In a preferred embodiment, the false nail removal device includes a cap 3. The cap 3 covers the nail removing part 2, like a pen cap.

### EXAMPLE 1

A false nail removal device includes a holding part 1 and a nail removing part 2, wherein the nail removing part 2 is arranged at one end of the holding part 1, and the nail removing part 2 is made of a hard material which is convenient for inserting into gaps between person's nails and artificial nails. During operation, the holding part 1 is held by an operator, the nail removing part 2 can be inserted into a gap between the person's nail and the artificial nail to remove the artificial nail.

### EXAMPLE 2

A false nail removal device includes a holding part 1 and a nail removing part 2, wherein the nail removing part 2 is arranged at one end of the holding part 1, and the nail removing part 2 is made of a hard material which is convenient for inserting into gaps between nails and artificial nails. The nail removing part 2 is provided with a channel 4 through which nail removing liquid passes. During operation, the holding part 1 is held by an operator, the nail removing part 2 can be inserted into the gap between the nail and the artificial nail, and the nail removing liquid enters the gap between the nail and the artificial nail through the channel 4 of the nail removing part 2, which is convenient for artificial nails removal.

### EXAMPLE 3

A false nail removal device includes a holding part 1 and a nail removing part 2, wherein the nail removing part 2 is arranged at one end of the holding part 1, and the nail removing part 2 is made of a hard material which is convenient for inserting into gaps between nails and artificial nails. One end of the nail removing part 2 far away from the in holding part 1 is provided with a slope 5. The slope 5 is convenient for inserting. The slope 5 facilitates the nail removing part 2 to be inserted into gaps between nails and artificial nails, whereby user comfortability is improved during the process of removing the nail.

### EXAMPLE 4

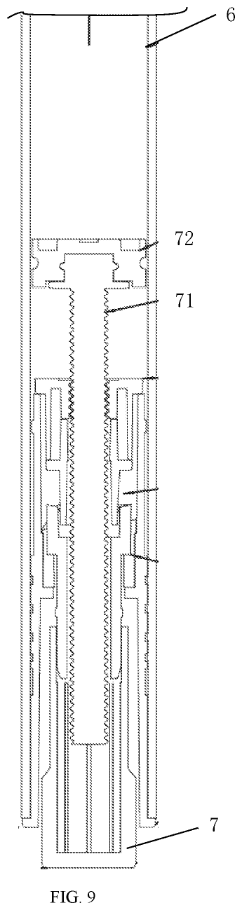

A false nail removal device includes a holding part 1 and a nail removing part 2, wherein the nail removing part 2 is arranged at one end of the holding part 1, and the nail removing part 2 is made of a hard material which is convenient for inserting into gaps between nails and artificial nails. The nail removing part 2 is provided with a channel 4 through which nail removing liquid passes. The end of the nail removing part 2 far away from the holding part 1 is provided with a slope 5, which is convenient for insertion. The holding part 1 is provided with a pressing device 7. The pressing device 7 may comprise a push rod 72 and a plunger 74 located at an end of the cavity 6, as shown in FIG. 9. During operation, the holding part 1 is held, the nail removing part 2 is inserted into the gap between the nail and the artificial nail. When the pressing device 7 is pressed, the nail removing liquid contained in the cavity 6 is pushed out to enter the gap between the nail and the artificial nail through the channel 4 of the nail removing part 2, which is convenient for nail removal.

### EXAMPLE 5

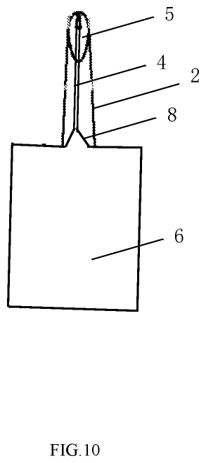

A false nail removal device includes a holding part 1 and a nail removing part 2, wherein the nail removing part 2 is arranged at one end of the holding part 1, and the nail removing part 2 is made of a hard material which is convenient for inserting into gaps between nails and artificial nails. The nail removing part 2 is provided with a channel 4 through which nail removing liquid passes. The end of the nail removing part 2 far away from the holding part 1 is provided with a slope 5, which is convenient for insertion. The holding part 1 is provided with a cavity 6 for storing the nail removing liquid and a pressing device 7. A linkage device 8 (shown in FIG. 10) is arranged between the cavity 6 and the nail removing part 2. During operation, the holding part 1 is held, the nail removing part 2 is inserted into the gap between the nail and the artificial nail. When the pressing device 7 is pressed, the linkage device 8 is driven to communicate the channel 4 with the cavity 6, and the nail removing liquid contained in the cavity 6 is pushed out to enter the gap between the nail and the artificial nail through the channel 4 of the nail removing part 2, which is convenient for nail removing. The linkage device 8 may be a one-way valve which is closed at a normal state (not working state) and is open when the pressing device 7 is pushed to cause the nail removing liquid in the cavity 6 to exert a force on the linkage device 8.

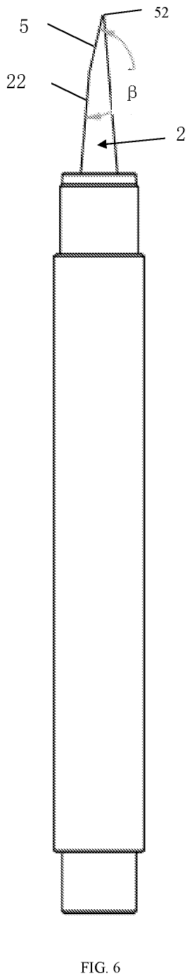

Referring to FIG. 6 which is a left side view of the device, preferably, the nail removing part 2 comprises an extension section 22 extending from the slope 5 in a direction away from the tip 52 of the nail removing part 2. The angle β formed between the extension section 22 and the slope 5 is equal to or greater than 170 degrees, preferably 172 degrees.

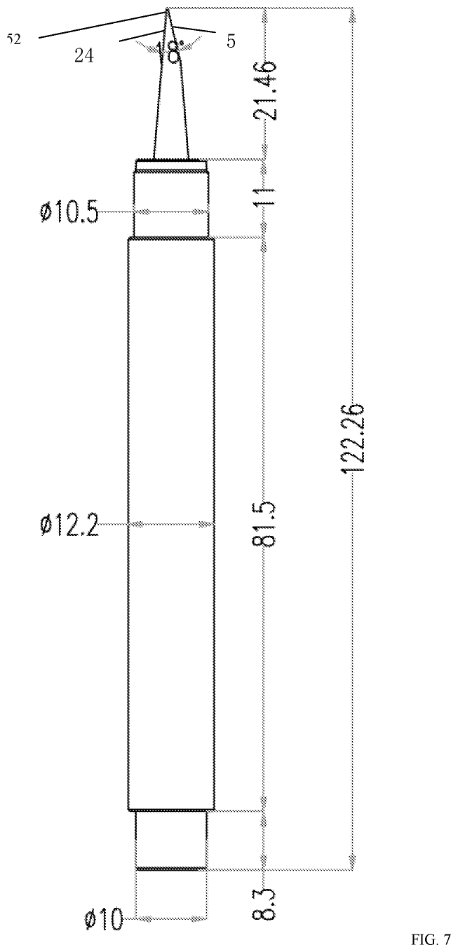

Referring to FIG. 7 which is a right side view of the device, preferably, the tip 52 has an angle which is equal to or less than 20 degrees, preferably 18 degrees. The angle of the tip 52 is formed between the slope 5 and the extension section 24 opposing the slope 5.

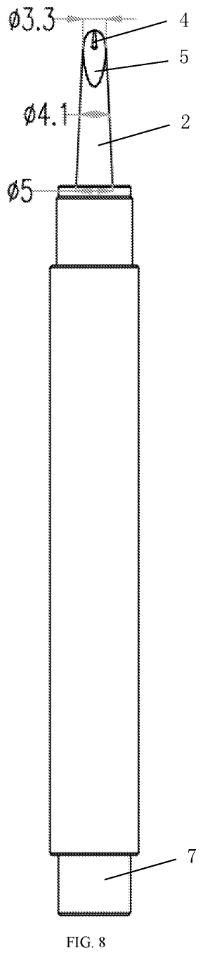

Referring to FIG. 8 which is a front view of the device, preferably, the diameters of the two ends and the middle point of the nail removing part 2 are 3.3, 4.1 and 5 mm respectively.

The above is only the preferred embodiment of this application, and it is not intended to limit this application. Any modification, equivalent substitution and improvement made within the spirit and principle of this application shall be included in the protection scope of this application.

What is claimed is:

1. A false nail removal device, comprising a holding part and a nail removing part, wherein the nail removing part is arranged at an end of the holding part, and the nail removing part is made of a hard material so that the nail removing part can be inserted between a person's nail and a false nail mounted on the person's nail.

2. The false nail removal device according to claim 1, wherein the nail removing part is provided with a channel.

3. The false nail removal device according to claim 1, wherein the nail removing part comprises a slope arranged at an end thereof opposite to the holding part and an extension section arranged at another end thereof close to the holding part; and
an angle formed between the slope and the extension section is equal to or greater than 170 degrees.

4. The false nail removal device according to claim 3, wherein the extension section has a height greater than that of the slope.

5. The false nail removal device according to claim 1, wherein the holding part is provided with a cavity for storing a nail removing liquid.

6. The false nail removal device according to claim 1, wherein the holding part is provided with a pressing device.

7. The false nail removal device according to claim 2, wherein a linkage device is arranged between the cavity and the nail removing part to selectively turn on or off communication between the cavity and the channel.

8. The false nail removal device according to claim 1, comprising a cap detachably sleeved on the nail removing part.

9. The false nail removal device according to claim 1, wherein a diameter of a middle point of the nail removing part is 4.1 mm, a diameter of an end of the nail removing part is 3.3 mm, and a diameter of an opposite end of the nail removing part is 5 mm.

10. The false nail removal device according to claim 3, wherein the nail removing part comprises a tip, and in a right side view of the device, the tip has an angle equal to or less than 20 degrees.
